(19) United States
(12) Patent Application Publication
(10) Pub. No.: US 2005/0228251 A1
(43) Pub. Date: Oct. 13, 2005

(54) System and method for displaying a three-dimensional image of an organ or structure inside the body

(75) Inventors: Mark L. Grabb, Burnt Hills, NY (US); Curtis G. Neason, New York, NY (US); Cynthia E. Landberg, Clifton Park, NY (US)

Correspondence Address: GE MEDICAL SYSTEM;C/O FOLEY & LARDNER, 777 EAST WISCONSIN AVENUE, MILWAUKEE, WI, 53202-5367, US

(73) Assignee: General Electric Company

(21) Appl. No.: 10/813,375

(22) Filed: Mar. 30, 2004

(51) Int. Cl. A61B 5/05

(52) U.S. Cl. 600/407

(57) ABSTRACT

A system for displaying a three-dimensional image of an organ or structure inside the body includes a processor configured to be communicatively coupled to a probe, the probe being configured to be located in or adjacent to the organ or structure inside the body. The system also includes memory coupled to the processor and configured to store image data pertaining to the organ or structure inside the body. The system also includes a three-dimensional display coupled to the processor and configured to simultaneously display the three-dimensional image and a representation of the probe.

### BACKGROUND OF THE INVENTION

The present description relates generally to systems and methods for displaying a three-dimensional image of an organ or structure inside the body. In particular, the present description relates to a system and method for displaying a three-dimensional image of an organ or structure inside the body in combination with an image-guided intervention procedure.

Presently, interventional procedures are used to diagnose and treat many medical conditions percutaneaously (i.e., through the skin) that might otherwise require surgery. Interventional procedures may include the use of probes such as, for example, balloons, catheters, microcatheters, stents, therapeutic embolization, etc. Many interventional procedures are conducted under image guidance, and the number of procedures conducted under image-guidance is growing. For example, today's interventional procedures are utilized in areas such as cardiology, radiology, vascular surgery, and biopsy. The use of image guidance allows interventional procedures to be less invasive than in the past. For example, today's electrophysiology (EP) procedures can be used to diagnose and/or treat a number of serious heart problems, and have replaced open-heart surgeries in many instances.

While EP procedures are classified as invasive cardiology, these procedures are minimally invasive with respect to open-heart surgery as an alternative. In a typical EP procedure, a probe such as catheter, (e.g., electrode catheter, balloon catheter, etc.) is inserted into a vein or artery and guided to the interior of the heart. Once inside the heart, the probe is contacted with the endocardium at multiple locations. At each location, the position of the catheter and the electrical properties of the endocardium can be measured. The attending physician can use this data to assist in locating the origin of, for example, a cardiac arrhythmia. The results of the EP study may lead to further treatment, such as the implantation of a pacemaker or implantable cardioverter defibrillator, or a prescription for antiarrhythmic medications. Oftentimes, however, the physician ablates (e.g., RF ablation, etc.) the area of the heart causing the arrhythmia immediately after diagnosing the problem. Generally, ablating an area of the heart renders it electrically inoperative, thus removing stray impulses and restoring the heart's normal electrical activity.

Many interventional procedures require sensing of the patient using multiple imaging technologies during the procedure. For example, one or more imaging devices (e.g., computed tomography (CT), magnetic resonance (MR), etc.) may be used to collect pre-operative imaging data before the procedure for interventional planning, and one or more other imaging devices (e.g., fluoroscope, ultrasound, etc.) may be used during the EP procedure to provide intra-operative imaging data. The intra-operative imaging device, however, may not provide a sufficient view of the anatomy and/or probes sufficient for real-time guidance and data collection during the interventional procedure, while the pre-operative data may not be sufficiently updated to reflect the patient's anatomy during the procedure. Further, the intra-operative imaging data and the pre-operative imaging data may need to be viewed as a whole for data collection and probe guidance during the intervention procedure.

Additionally, many other devices may be used to collect data to monitor the patient during the intervention procedure. For example, body surface electrocardiogram (ECG) data may be collected during the intervention procedure. Probes (e.g., catheters) may-be inserted into the heart to collect more localized ECG data by measuring the electrical activity. Further, navigational systems providing location data may be used to track the locations and orientations of the probes during the intervention procedure. Today, much of this data is presented to the interventionalist via flat displays, and the data is not presented to the interventionalist in a way that aids him or her to efficiently and effectively plan, manage and/or perform an intervention procedure. Thus, there is a need for an improved system and method for displaying an image of an organ or structure inside the body.

### SUMMARY OF THE INVENTION

According to a first exemplary embodiment, a system for displaying a three-dimensional image of an organ or structure inside the body includes a processor configured to be communicatively coupled to a probe, the probe being configured to be located in or adjacent to the organ or structure inside the body. The system also includes memory coupled to the processor and configured to store image data pertaining to the organ or structure inside the body. The system also includes a three-dimensional display coupled to the processor and configured to simultaneously display the three-dimensional image and a representation of the probe.

According to a second exemplary embodiment, a system for displaying a three-dimensional image of a heart includes a processor configured to be communicatively coupled to a probe. The system also includes memory coupled to the processor and configured to store image data pertaining to the heart. The system also includes a three-dimensional display coupled to the processor and configured to simultaneously display the three-dimensional image of the heart and a representation of the probe.

According to a third exemplary embodiment, a system for displaying a three-dimensional image of an organ or structure inside the body includes a processor configured to be communicatively coupled to a probe, the probe being configured to be located in or adjacent to the organ or structure inside the body and to collect data representative of the electrical properties of the organ or structure inside the body. The system also includes memory coupled to the processor and configured to store image data pertaining to the organ or structure inside the body. The system also includes a three-dimensional display coupled to the processor and configured to display the three-dimensional image and a map of the electrical properties of the organ or structure inside the body.

According to a fourth exemplary embodiment, a method for displaying a three-dimensional image of an organ or structure inside the body includes acquiring a three-dimensional image of the organ or structure inside the body, registering a representation of a probe with the three-dimensional image, the probe being located in or adjacent to the organ or structure inside the body, and simultaneously displaying a representation of the probe with the three-dimensional image using a three-dimensional display.

According to a fifth exemplary embodiment, a system for displaying a three-dimensional image of an organ or structure inside the body includes memory configured to store a first set of image data pertaining to the organ or structure inside the body. The system also includes a processor coupled to the memory and configured to be communicatively coupled to an imaging device and a probe, the imaging device being configured to generate a second set of image data pertaining to the organ or structure inside the body, and the probe being configured to be located in or adjacent to the organ or structure inside the body. The processor is further configured to generate the three-dimensional image using the first set of image data and the second set of image data. The system also includes a three-dimensional display coupled to the processor and configured to simultaneously display the three-dimensional image and a representation of the probe.

### BRIEF DESCRIPTION OF THE DRAWINGS

FIG. 1 is a diagram of a system for displaying a three-dimensional image of an organ or structure inside the body according to an exemplary embodiment.

FIG. 2 illustrates a three-dimensional image displayed in a three-dimensional display according to an exemplary embodiment.

FIG. 3 is a flow diagram depicting a method for displaying a three-dimensional image of an organ or structure inside the body using the system of FIG. 1 according to an exemplary embodiment.

FIG. 4 is a flow diagram depicting a method for using the system of FIG. 1 in an image guided intervention procedure according to an exemplary embodiment.

### DETAILED DESCRIPTION

Turning now to the FIGURES which illustrate exemplary embodiments, a system and method for displaying a three-dimensional (3D) image (e;g., volumetric, etc.) of an organ or structure inside the body are shown. A 3D image is displayed which is representative of an organ or structure inside the body. The 3D image may be simultaneously displayed with a 3D representation of a probe inside the body which has been, for example, registered with the 3D image. Additionally, the 3D image may be simultaneously displayed with other data or information related to the intervention procedure which may also be registered with the 3D image. The other data or information may include, for example, color changes to the 3D image to indicate electrical measurements or other functional data related to the organ or structure inside the body, or historical data such as locations of previous electrical measurements or locations of lesions on the myocardium resulting from an ablation procedure. Other information may also include auxiliary data, such as graphs or numbers to aid in the intervention procedure, including, for example, blood pressure or body surface electrocardiogram (ECG) data, or workflow instructions. Other information may further include visual navigational information for use during the intervention procedure, such as changes in color of a target location to indicate the quantitative proximity of a probe to a target location. Further, the validity of the 3D image of the organ or structure inside the body may be verified during the intervention procedure, and if it is necessary to generate a new 3D image, a warning may be visually displayed with the current 3D image. Similarly, warnings of unreliable location data with respect to the 3D representation of the probe inside the body may also be provided.

The present description is generally provided in the context of displaying a 3D image of an organ or structure inside the body. Although the present description is provided primarily in the context of simultaneously displaying a 3D image of the heart with a representation of a catheter which is inside the heart, it should be understood that the systems and methods described and claimed herein may also be used in other contexts. For example, one or more images of other organs (e.g., brain, liver, etc.) of a human or, broadly speaking, animal body, may be utilized. Further, probes other than a catheter, (e.g., biopsy needle, etc.) may be used. Additionally, other types of data or information than those disclosed herein may be incorporated into the 3D image. Accordingly, the systems and methods described herein are widely applicable in a number of other areas beyond what is described in detail herein. Also, it should be understood that although oftentimes a single 3D image of an organ or structure inside the body is simultaneously displayed with a single representation of a probe, one or more 3D images may be registered with one or more representations of one or more probes. It should also be understood that a particular example or embodiment described herein may be combined with one or more other examples or embodiments also described herein to form various additional embodiments. Accordingly, the systems and methods described herein may encompass various embodiments and permutations as may be appropriate.

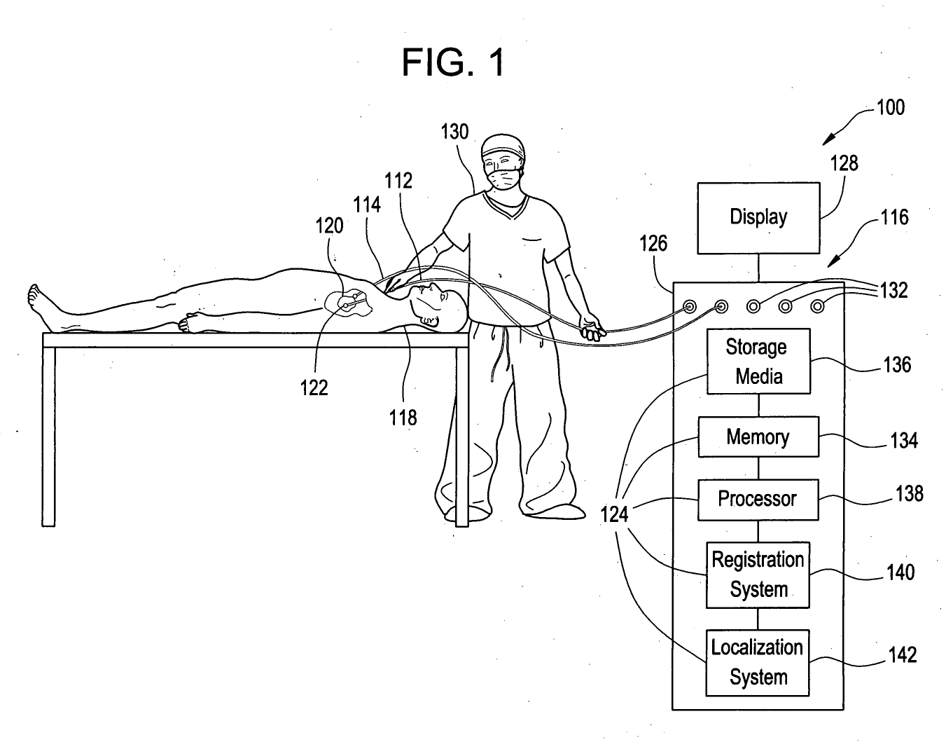

FIG. 1 illustrates a system 100 according to an exemplary embodiment. System 100 may include a probe 112, an imaging device 114, and a console or computer 116. System 100, broadly described, may be used to simultaneously display a 3D image of an organ or structure inside the body and a representation of a probe 112 inside the body for the purpose of indicating where probe 112 is located with respect to the organ or structure inside the body. The term “representation” as used herein should be given its ordinary and accustomed meaning. However, regardless of its ordinary and accustomed meaning, the term “representations should not be construed to require the representation to be in any way similar in size, shape, etc. (although they may be similar in size, shape, etc.) as the thing being represented (e.g., a square is used to represent probe 112 even though probe 112 is not the shape or size of a square). In particular, system 100 may be used to simultaneously display a 3D image of an organ or structure inside the body and a representation of probe 112 with respect to the organ or structure inside the body, wherein the representation of probe 112 has been spatially and/or temporally registered with the 3D image.

System 100 may be a wide variety of systems used for an equally wide variety of interventional procedures. For example, in one embodiment, system 100 may be any system that is configured to use probe 112 to measure, monitor, diagnose, manipulate, or otherwise provide information about an organ or structure inside the body. In another embodiment, system 100 may be an EP monitoring system that is configured to use a probe to purposefully alter or provide information regarding the electrical activity of an organ or structure inside the body. In another embodiment, system 100 may be a cardiac EP monitoring system. In general, the cardiac EP monitoring system may be configured to provide information about or purposefully alter the electrical activity of a heart using an probe which is in or adjacent to the heart.

System 100 may also be configured to include additional components and systems. For example, system 100 may further comprise a printer. System 100 may also be configured as part of a network of computers (e.g., wireless, cabled, secure network, etc.) or as a stand-alone system. In one embodiment, system 100 may comprise an ECG monitoring system. The ECG monitoring system may be a conventional twelve lead ECG monitoring system. In other embodiments, the ECG monitoring system may include any suitable and/or desirable configuration of leads, etc. to provide the information necessary for the particular use of system 100. In another embodiment, system 100 may comprise a system to monitor the blood pressure of patient 118. This may be a conventional blood pressure monitoring system or may be a system that monitors the blood pressure using a transducer placed on or adjacent to a vein or artery. In short, there are a number of conventional systems and components that may also be included as part of system 100.

Probe 112 is communicatively coupled to console or computer 116 and may be any number of devices typically employed in an image-guided intervention procedure. In general, probe 112 may be located in or adjacent to an organ or structure inside the body, such as a heart 120 (shown in FIG. 1 in a cross-sectional view to expose probe 112) of patient 118. For example, probe 112 may be a catheter, biopsy needle, trocar, implant, etc. In one embodiment, probe 112 may include one or more sensors 122, which are configured to sense the electrical properties (e.g., electrical potential at one or more locations of the endocardium, activation times, etc.) of heart 120. The electrical properties may then be communicated back to console 116 and displayed on display 128. In an exemplary embodiment, probe 112 may comprise a plurality of sensors configured to sense the electrical properties of heart 120 (e.g., probe 112 is a balloon catheter, etc.). In another embodiment, multiple probes 120 may be used that each comprise one or more sensors configured to sense the electrical properties of heart 120.

Imaging device 114 is communicatively coupled to console or computer 116 and may be any number of suitable 3D imaging devices utilizing a variety of configurations and/or imaging technologies. For example, imaging device 114 may be a CT device, ultrasound device, x-ray device, MR device, etc. Imaging device 114 may also be an internal or an external medical imaging device, such as an intra-cardiac ultrasound device or an extra-cardiac ultrasound device. Imaging device 114 provides image data to system 100 which may be used to generate one or more 3D images to be stored, manipulated, and or displayed. For example, in one embodiment, imaging device 114 may be a CT device which provides “pre-operative” image data to system 100 prior to the intervention procedure to be displayed in the form of a 3D image representative of the position of heart 120 during one phase of the heartbeat cycle of patient 118. Output from imaging device 114 may also include “intra-operative” image data generated continuously or periodically throughout the intervention procedure to be used by system 100 in conjunction with, for example, pre-operative image data, to generate the 3D image. For example, in one embodiment, imaging device 114 may be an ultrasound device which provides continuous or periodic intra-operative real time image data to system 100 throughout the image-guided intervention procedure to modify or supplement (e.g., by using a deformable registration system as will be described below) pre-operative image data generated prior to the image-guided intervention procedure using CT technology. As will be described below, image data from imaging device 114 may further be used by system 100 to register a 3D image of an organ or structure inside the body with a representation of probe 112.

Console or computer 116 is communicatively coupled to probe 112 and imaging device 114 and includes computer components 124 in cabinet 126, and display 128. Information sensed by probe 112 and imaging device 114 may be communicated to computer components 124. Information from computer components 124 may be communicated to display 128 where it is displayed to a nearby person 130 (e.g., interventionalist, attending physician, nurse, technician, etc.). The configuration shown in FIG. 1 is only one of many suitable configurations. For example, in another embodiment, probe 112 and/or imaging device 114 may be communicatively coupled directly to display 128. In this embodiment, display 128 may be configured to display the information provided by probe 112 and/or imaging device 114 without the information being communicated through cabinet 126 (e.g., display 128 comprises the necessary computer components 124 to receive information from probe 112 and/or imaging device 114). In another embodiment, display 128 may be combined with cabinet 126 so that the functions generally performed by computer components 124 in cabinet 126 and display 128 are performed by the combined unit (e.g., display 128 comprises all of computer components 124). In another embodiment, console 116 may include two or more displays 128. In one embodiment, display 128 may be configured to be in a location that is convenient for person 130 to view (e.g., at height of person 130's eyes as person 130 is standing, etc.) as person 130 manipulates probe 112. In one embodiment, console 116 is a desktop computer. In another embodiment, console 116 may be configured to include input locations 132 on cabinet 126 or display 128 that are configured to receive additional information pertaining to patient 118. For example, in one embodiment, input locations 132 may include one or more input locations configured to receive input from ECG leads, etc.

Computer components 124 in cabinet 126, shown in FIG. 1, may comprise a memory 134, storage media 136, a processor 138, a registration system 140, a localization system 142, and one or more input devices (e.g., keyboard, mouse, etc.). Cabinet 126 is configured to receive information from probe 112 and imaging device 114, process the information, and provide output using display 128. The information provided to cabinet 126 may be continually stored (i.e., all information is stored as it is received) or intermittently stored (i.e., periodic samples of the information are stored) using memory 134 or storage media 136 (e.g., optical storage disk (e.g., CD, DVD, etc.), high performance magneto optical disk, magnetic disk, etc.) for later retrieval. Processor 138 may include a single processor, or one or more processors communicatively coupled together and configured to carry out various tasks as required by system 100. Processor 138 may also be communicatively coupled with and operate in conjunction with other systems either internal or external to system 100, such as localization system 142 or registration system 140.

Registration system 140 may be used, for example, to register intra-operative image data from imaging device 114 with pre-operative image data to generate the 3D image. In one embodiment, registration system 140 may be a deformable registration system. The deformable registration system may be used, for example, to generate a 3D image by deformably combining intra-operative image data from imaging device 114 with pre-operative image data. In one exemplary embodiment, the deformable registration system is used to generate the 3D image wherein pre-operative image data generated using CT technology is weighted and deformed to match 3D continuous or periodic intra-operative image data provided to system 100 from imaging device 114 during the intervention procedure, where imaging device 114 is an ultrasound imaging device. The use of deformable registration system 140 in conjunction with system 100 to combine intra-operative ultrasound image data with pre-operative CT image data provides the advantages of high resolution, high contrast CT imaging technology prior to the procedure, as well as the advantage of being an updated representation of the organ or structure inside the body during the intervention procedure. In another embodiment, registration system 140 may be further configured to compare the continuous or periodic intra-operative image data from imaging device 114 with the pre-operative image data during the procedure, and to provide a warning or alarm in conjunction with system 100 when the intra-operative image data differs from the pre-operative image data according to a predetermined criterion. Using this enhanced configuration, system 100 may determine that, for example, a new 3D image should be generated and display a warning.

System 100 may further include localization system 142. Localization system 142 may be used, e.g., continuously or periodically, to determine the location of probe 112, as well as the location of imaging device 114, where these devices may be configured to be located by localization system 142, and to register these devices to the same coordinate system with respect to a global position. Localization system 142 may then be used to register an organ or structure inside the body (e.g., heart 120) in the same coordinate system. Any suitable localization system, such as a system utilizing electromagnetic (EM) tracking technology, may be used as would be recognized by those of ordinary skill. In one exemplary embodiment, an EM localization system may be utilized by system 100 to locate imaging device 114, where imaging device 114 is an ultrasound device, as well as to locate one or more probes 112 inserted in heart 120 with respect to a global position, thus registering the locations of these devices with the global position. The intra-operative image data from ultrasound imaging device 114 contains sufficient detail of heart 120 to then enable localization system 142 to register the location of heart 120 with respect to the same global position, thus registering heart 120, ultrasound imaging device 114, and the probe(s) 112 in the same coordinate system. In another exemplary embodiment, the EM localization system may be further configured to continuously or periodically estimate the location of each probe 112 using continuously or periodically updated image data from imaging device 114, and to optimize this location estimate with continuous or periodically updated location data from each individual intervention device 112. In another exemplary embodiment, the EM localization system may be further configured to provide a warning in conjunction with system 100 when the estimate of the location of each probe 112 obtained from the intra-operative image data from imaging device 114 differs from the location data from each individual probe 112 according to a predetermined criterion. Using this enhanced configuration, system 100 may detect unreliable location data from imaging device 114 and/or one or more probes 112 and display a warning.

Localization system 142 may further be used in conjunction with registration system 140 to, for example, continuously or periodically register a representation of one or more probes 112 with a 3D image. In one embodiment, registration system 142 may be used to register pre-operative image data with intra-operative image data to generate the 3D image. Localization system 142 may be used to continuously or periodically locate imaging device 114, probe 112, and, for example, heart 120. In this way, the location of heart 120 (and the corresponding intra-operative image data used by localization system 142 to locate heart 120), imaging device 114, and probe 112 are all registered in the same coordinate system, and the intra-operative image data is registered with and incorporated into the 3D image. System 100 may then use this information to continuously or periodically register a representation of probe 112 with the 3D image spatially and/or temporally by weighing the location data from localization system 142 with the 3D image. In one embodiment, the 3D image comprises a series of 3D images, each representative of a different phase in the heartbeat cycle of patient 118, and localization system 142 samples the location data at the heart rate of patient 118 to correspond to each phase represented in the 3D image. A representation of probe 112 may then be registered with each phase image contained in the 3D image.

Display 128 is a 3D display and may be configured to provide output to a user in the form of information, which may include alphanumeric (e.g., text, numbers, etc.) output, graphical image output, etc. Display 128 may be any number of suitable 3D displays in a number of suitable configurations. For example, in one embodiment, display 128 is a spatial 3D display, such as the 3D display manufactured by Actuality Systems, Inc. under the PERSPECTA trademark. The term “spatial 3D display” refers to a display wherein the 3D image physically occupies a region in space, as compared with a stereoscopic 3D display, wherein, for example, images of an object seen from slightly dissimilar viewpoints are combined to render a 3D appearance in two dimensions. In one embodiment, display 128 may be configured to display one or more 3D images of an organ or structure inside the body. Desirably, display 128 may be configured to display 3D images based on image data acquired using CT, MR, x-ray, and/or ultrasound imaging technologies.

Display 128 may also be configured to simultaneously display one or more representations of one or more probes 112 with a 3D image. Any suitable marker or identifier may be used to represent probe 112 on display 128. For example, the representation may be a scaled replica of probe 112, or may be another predetermined shape, size, color, etc. In one embodiment, display 128 may be configured to display a representation of the location of probe 112 with respect to heart 120. In another embodiment, one or more probes 112, imaging device 114, and heart 120 may be located with respect to a global position and further registered with a 3D image representative of heart 120, and display 128 may be configured to simultaneously display the 3D image and representations of the one or more probes 112 with respect to heart 120, for the purpose of indicating where each probe 112 is located with respect to heart 120 during an intervention procedure. In another embodiment, each representation may be continuously or periodically registered with the 3D image to indicate the current location of each probe 112 during the intervention procedure. In this manner, person 130 is able to observe display 128 to determine the location of probe 112 inside heart 120. Person 130 may then adjust and manipulate probe 112 accordingly, while observing the progress via display 128.

Display 128 may also be configured to display other data sources and information relevant to an intervention procedure with a 3D image. The other data or information may include, for example, color changes to the 3D image to indicate electrical measurements or other functional data related to the organ or structure inside the body, or historical data such as locations of previous electrical measurements or locations of lesions on the myocardium resulting from an ablation procedure. Other information may also include auxiliary data, such as graphs or numbers to aid in the intervention procedure, including, for example, blood pressure or body surface electrocardiogram (ECG) data, or workflow instructions. Other information may further include visual navigational information for use during the intervention procedure, such as changes in color of various locations or areas of the 3D image to indicate the quantitative proximity of probe 112 to the location or area. Any combination of these data sources or information may be simultaneously displayed with the 3D image.

For example, in one embodiment, display 128 may be configured to display functional data related to an organ or structure inside the body with the 3D image. Specifically, in one embodiment the functional data may include electrical properties of heart 120, which in turn may include, for example, intra-cardiac or body surface electrocardiogram (ECG) data. In one embodiment, the electrical properties may be sensed by probe 112 (e.g., probe 112 is a catheter configured to collect intra-cardiac ECG measurements). In another embodiment, the electrical properties may be calculated, for example, based on a cardiac model which relates body surface ECG measurements to intra-cardiac cell-level activity. In another embodiment, probe 112 may be a catheter configured to collect intra-cardiac ECG data from heart 120, and display 128 may be further configured to simultaneously display an image of heart 120, a representation of probe 112, and a map of the electrical properties of heart 120, all of which may be registered to each other. In yet another embodiment, the representation of probe 112 may be continuously or periodically registered with the 3D image and displayed in display 128, and the electrical properties of heart 120 may further be registered with the 3D image to generate the map displayed in display 128 as each measurement is taken. The electrical properties may be displayed in any number of ways by display 128. In one embodiment, the electrical properties are color coded onto the 3D image in display 128 so that person 130 can observe the electrical properties of various areas of heart 120 in display 128 as the electrical measurements are taken.

In another embodiment, display 128 may be further configured to display historical data related to the intervention procedure with the 3D image. Historical data may include, for example, previous ECG measurements and locations, and previous ablation sites. In one embodiment, historical data related to locations where ablations of heart 120 have been made by probe 112 (e.g., probe 112 is a catheter) is provided to system 100, and display 128 may be further configured simultaneously display an image of heart 120, a representation of probe 112, and representations of the locations of the ablations of heart 120, all of which may be registered to each other. The historical information may by indicated in display 128 in any number of ways. For example, in one embodiment the ablation locations of heart 120 may be indicated by, for example, changes in color of the corresponding location on the 3D image. In this manner, person 130 is able to observe display 128 to determine which locations have already been ablated by probe 112. Person 130 may then adjust and manipulate probe 112 accordingly, while observing the progress in display 128.

In another embodiment, display 128 may be further configured to display auxiliary data related to the intervention procedure with the 3D image. Auxiliary data may include, for example, charts, graphs, or other related data such as blood pressure or body surface ECG information, to aid in the intervention procedure. Other examples of auxiliary data which may be displayed on display 128 may include workflow instructions for the intervention procedure, duration of the procedure, local time, and other additional information related to patient 118.

Auxiliary data may also include warnings provided by system 100. Auxiliary data in the form of a warning provided by system 100 may include various visual formats (e.g., color, text, graphics, etc.). For example, in one embodiment, system 100 may provide warnings in the form of color changes to the 3D image. In another embodiment, system 100 may provide warnings in the form of text messages and/or correlation data related to one or more data sources. Auxiliary data in the form of a warning provided by system 100 may also include various audible formats where system 100 is configured to provide an audio output.

In one embodiment, system 100 may be configured to provide a warning when continuous or periodic intra-operative image data from imaging device 114 differs from pre-operative image data according to a predetermined criterion. In another embodiment, system 100 may be configured to provide a warning when an estimate of the location of each probe 112 obtained from the intra-operative image data from imaging device 114 differs from the location data from each individual probe 112 according to a predetermined criterion. In another embodiment, system 100 may be configured to provide a warning when data from another data source (e.g., ECG data, respiratory measurements, blood pressure readings, etc.) differs from the location data or image data. For example, in one embodiment, ECG data may be monitored and aligned with the location data of a probe 112 adjacent to heart 120, and system 100 may be configured to provide a warning when the ECG data differs from the location data according to a predetermined criterion. In another embodiment, ECG data may be monitored and aligned with intra-operative image data of heart 120 from imaging device 114, and system 100 may be configured to provide a warning when the ECG data differs from the location data according to a predetermined criterion.

In another embodiment, display 128 may be configured to display visual navigational information such as, for example, information indicating the proximity of probe 112 to a particular location or area in an organ or structure inside the body. For example, in one embodiment, display 128 may be configured to simultaneously display a 3D image of heart 120, a representation of the location of probe 112 with respect to heart 120, and a visual indication of the proximity of probe 112 with respect to various locations or areas in heart 120, all of which are registered to each other. The visual navigational information may by indicated by display 128 in any number of ways. For example, in one embodiment the quantitative proximity of probe 112 (e.g., a catheter) to a particular location or area may be indicated by, for example, changes in color of the location or area on the 3D image of heart 120. In this manner, person 130 is able to observe display 128 to determine the location of probe 112 inside heart 120 with respect to the location or area. Person 130 may then adjust and manipulate probe 112 accordingly, while observing the progress in real time in display 128. Of course, in addition to the embodiments specifically described, display 128 may be configured to display any suitable combination of a 3D image, a representation of probe 112, and other data sources and information (e.g., electrical properties of heart 120, etc.), any of which may be registered and/or simultaneously displayed with each other.

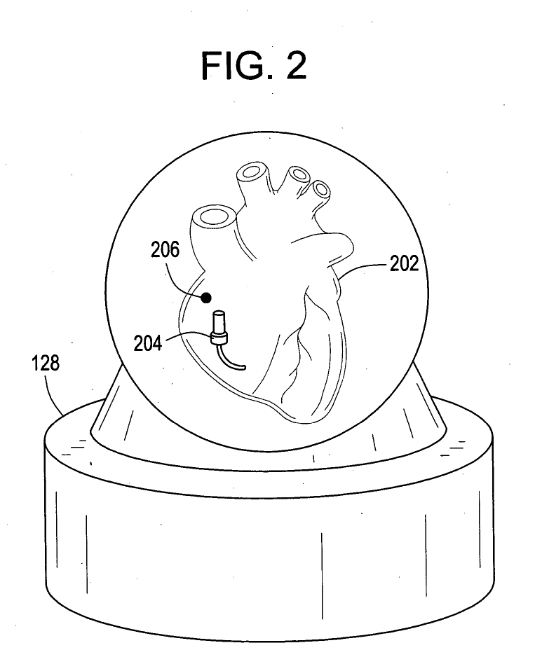

FIG. 2 illustrates a three-dimensional image 202 displayed in a three-dimensional display 128 according to an exemplary embodiment. In the illustrated embodiment, display 128 is a spatial three-dimensional display, while 3D image 202 is a three dimensional image of heart 120 (shown in FIG. 1). Also shown in FIG. 2 is a representation 204 of probe 112 (shown in FIG. 1) which is located adjacent to the heart.

3D image 202 may be based on, for example, image data from CT, MR, x-ray, and/or ultrasound imaging devices, and may be based in part on computer simulation or a standard computer model. Further, 3D image 202 may be based on pre-operative image data, intra-operative image data, or may be a combination of both (e.g., using deformable registration technology). For example, in one embodiment, 3D image 202 may first be generated prior to the intervention procedure using pre-operative image data. Typically, in embodiments where 3D image 202 is based on CT or MR image data, the image data may first be acquired as pre-operative image data prior to probe 112 being inserted into a patient or before an interventional procedure (e.g., an EP monitoring procedure) is initiated. The pre-operative image data may then be modified or supplemented with intra-operative image data from imaging device 114 (shown in FIG. 1) generated immediately prior to and/or during the intervention procedure to generate 3D image 202.

3D image 202 may consist of a single image or may consist of a series of images. In one exemplary embodiment, 3D image 202 comprises a series of 3D images representative of a different phase in the heartbeat cycle of patient 118 (shown in FIG. 1). 3D image 202 may further incorporate additional segmentation and modeling in order to accurately define the organ or structure inside the body. 3D image 202 may also indicate one or more locations or areas 206 of clinical interest (e.g., sites for ECG measurements or catheter ablations).

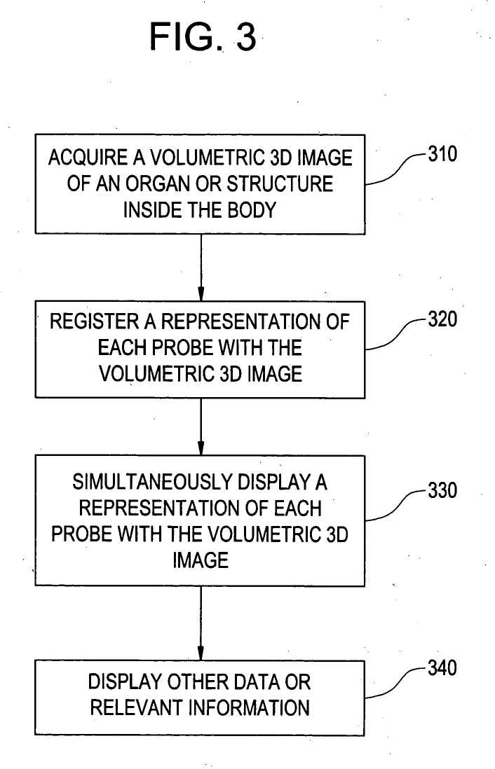

FIG. 3 illustrates a method for displaying a 3D image of an organ or structure inside the body using system 100 (shown in FIG. 1) according to an exemplary embodiment. At step 310, a 3D image of the organ or structure inside the body may be acquired. The 3D image may be composed of intra-operative image data, pre-operative image data, or both. In one exemplary embodiment, the 3D image may be generated from pre-operative imaging data (e.g., CT image data generated by imaging heart 120 prior to the intervention procedure) in combination with intra-operative imaging data from imaging device 114, (e.g., imaging device 114 is an ultrasound device located either internal or external to heart 120). In another embodiment, a deformable registration system is further utilized to generate the 3D image of heart 120. In yet another embodiment, the 3D image comprises a series of 3D images representative of heart 120 during a phase of the heartbeat cycle of patient 118.

At step 320, one or more probes 112 may be inserted into the organ or structure inside the body and a representation of each probe 112 may be registered with the 3D image. In one embodiment, probe 112 may be a catheter inserted into heart 120, wherein the catheter may be configured to collect ECG information as part of an EP procedure from various locations or areas of heart 120. In this embodiment, imaging device 114 may be located with respect to a global position using EM localization system 142. Further, probe 112 may be tracked with respect to the same global position using EM localization system 142. Through the common global position, the intra-operative ultrasound device 114 may be registered to the location of each probe 112. Further, imaging device 114 views a sufficient amount of heart 120 with sufficient temporal and spatial resolution and sufficient contrast to register the location of heart 120 with the global position using ultrasound device 114 and EM localization system 142. Accordingly, heart 120 may be registered in the same coordinate system as each probe 112.

Continuing with the embodiment, a representation of each probe 112 maybe registered with the 3D image using EM localization system 142 and registration system 140. The catheter and heart location data may be weighed with respect to the each of the phase images in the 3D image (e.g., the location data is sampled at the heart rate of patient 118 to correspond to each phase represented in the 3D image).

At step 330, the 3D image may be simultaneously displayed on display 128 with a representation of each probe 112 which has been registered with the 3D image. In this way, the location of each probe 112 with respect to the organ or structure inside the body may be indicated on display 128. In one embodiment, each representation may be continuously or periodically registered with the 3D image according to step 320 such that the current location of each probe 112 may be indicated on display 128.

At step 340, other data or information relevant to the intervention procedure may be displayed with the 3D image. In one embodiment, functional information related to the organ or structure inside the body may be displayed. In another embodiment, one or more probes 112 may collect intra-cardiac ECG information related to heart 120, and this electrical activity information may be color coded onto the 3D image. In another embodiment, historical data, auxiliary data, and/or visual navigational information may also be simultaneously displayed with the 3D image.

Steps 310 to 340 may be performed on a repeating basis as necessary throughout the procedure. For example, in one embodiment, system 100 may continuously or periodically register a representation of probe 112 with the 3D image and may further be configured to generate a warning or alarm to be displayed on display 128 when the intra-operative image data from imaging device 114 differs from the pre-operative image data according to a predetermined criterion. System 100 may then generate a new 3D image if necessary.

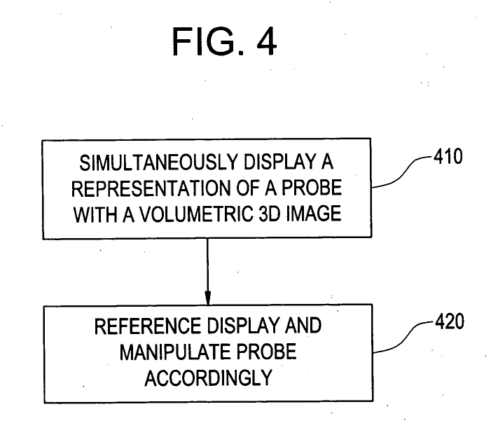

FIG. 4 illustrates a method for using system 100 (shown in FIG. 1) to perform an image guided intervention procedure according to an exemplary embodiment. At step 410, a 3D image of an organ or structure inside the body may be simultaneously displayed with a representation of a probe 112 according to the method shown in FIG. 3. For example, in one embodiment, a 3D image of heart 120 may be simultaneously displayed with a representation of probe 112, wherein probe 112 may be a catheter configured to collect electrical information from various locations or areas in heart 120 which may be indicated in the 3D image. In another embodiment, a map of the electrical properties of heart 120 may be simultaneously displayed with the 3D image as each electrical measurement is taken. In another embodiment, visual navigational information may be simultaneously displayed with the 3D image in the form of changes in color of each area or location to indicate the quantitative proximity of probe 112. Other combinations of relevant data or information may further be displayed with the 3D image.

At step 420, person 130 may reference display 128 and may manipulate probe 112 accordingly, while observing the progress. In one embodiment, person 130 may observe display 128 to determine the location of probe 112 inside heart 120 with respect to a location or area in heart 120 indicated in the 3D image. Referring to the 3D image in display 128, person 130 may adjust and manipulate probe 112 to the location or area of heart 120 while observing the progress on display 128. In one embodiment, when the visual navigational information indicates that the probe 112 has reached the location or area, an electrical measurement may be taken, and the completed electrical measurement may be indicated in the form of a change in color of the location or area indicated in the 3D image as part of a map of the electrical properties of heart 120. The map may then be used, e.g., to plan and perform a subsequent interventional procedure (e.g., a catheter ablation procedure).

System 100 may further be used as a user interface for planning or teaching, or used as a graphical user interface for commanding a semi-automated or fully automated interventional system. In one embodiment, system 100 may be used as a planning or teaching tool and may further include an input device (e.g., keyboard, mouse, etc.), and may be further configured to compute changes to the electrical or mechanical properties of the organ or structure inside the body based on, for example, planned catheter ablations in an intervention procedure entered by person 130 using the input device. As each step in the planned intervention procedure is entered, the resulting changes to the electrical or other properties may be used by person 130 to plan the next step of the intervention procedure. The specific workflow of the procedure may further be stored in memory and later be simultaneously displayed with a 3D image as auxiliary data to be viewed during the actual interventional procedure, keying person 130 as to the next step based on the interventional planning. In another embodiment, system 100 may further be used as a graphical user interface for commanding a semi-automated or fully automated interventional system, and may further include one or more user input devices, as well as one or more automated probes, such as an automated catheter configured to be controlled by system 100. Imaging device 114 may further be used to identify locations or areas for one or more of the automated catheters to be placed. Person 130 may then select one or more locations or areas using the input device. In response to the input information, the automated catheters may then move to the specified locations or area.

The system and method for displaying a 3D image of an organ or structure inside the body disclosed herein provides many advantages. It provides a 3D display of multiple data sources that enables an interventionalist or other user to efficiently and effectively navigate probes around the interior of the heart or other organ or structure inside the body during an intervention procedure, as well as to plan, manage, and otherwise perform an intervention procedure. The disclosed system and method may also reduce the amount of time required for an intervention procedure, limit the need for ionizing radiation throughout an intervention procedure, improve patient outcomes, and decrease a patent's length of stay in the hospital for complex EP procedures such as atrium fibrillation and biventricular pacemaker placement. The system and method may further decrease the likelihood of major complications during an interventional procedure by, for example, reducing the likelihood of puncturing a cardiac wall while manipulating a catheter or other probe.

The construction and arrangement of the elements described herein are illustrative only. Although only a few embodiments have been described in detail in this disclosure, it should be understood that many modifications are possible without materially departing from the novel teachings and advantages of the subject matter recited in the claims. Accordingly, all such modifications are intended to be included within the scope of the methods and systems described herein. The order or sequence of any process or method steps may be varied or re-sequenced according to alternative embodiments. Other substitutions, modifications, changes and omissions may be made in the design, operating conditions and arrangement of the embodiments without departing from the spirit and scope of the methods and systems described herein.

1. A system for displaying a three-dimensional image of an organ or structure inside the body, the system comprising:
a processor configured to be communicatively coupled to a probe, the probe being configured to be located in or adjacent to the organ or structure inside the body;
memory coupled to the processor and configured to store image data pertaining to the organ or structure inside the body; and
a three-dimensional display coupled to the processor and configured to simultaneously display the three-dimensional image and a representation of the probe.

2. The system of claim 1, wherein the representation of the probe is registered with the three dimensional image of the organ or structure inside the body.

3. The system of claim 1, wherein the representation of the probe is registered with the three dimensional image of the organ or structure inside the body using a localization system.

4. The system of claim 1, wherein the organ or structure inside the body is a heart.

5. The system of claim 1, wherein the probe is a catheter.

6. The system of claim 1, wherein the system is an electrophysiology system.

7. The system of claim 1, wherein the image data is acquired prior to the probe being positioned inside the body.

8. The system of claim 1, wherein the image data is acquired during the image-guided intervention procedure using an internal medical imaging device.

9. The system of claim 1, wherein the system is further configured to display a map of the electrical properties of the organ or structure inside the body.

10. The system of claim 1, wherein the system is further configured to display historical data related to the organ or structure inside the body.

11. The system of claim 1, wherein the system is further configured to display auxiliary data related to an image-guided interventional procedure.

12. The system of claim 1, wherein the display is further configured to display visual navigational information related to an image-guided intervention procedure.

13. The system of claim 1, wherein the three-dimensional display is a spatial three-dimensional display.

14. A system for displaying a three-dimensional image of a heart, the system comprising:
a processor configured to be communicatively coupled to a probe;
memory coupled to the processor and configured to store image data pertaining to the heart; and
a three-dimensional display coupled to the processor and configured to simultaneously display the three-dimensional image of the heart and a representation of the probe.

15. The system of claim 14, wherein the representation of the probe is registered with the three dimensional image of the heart.

16. The system of claim 14, wherein the representation of the probe is registered with the three dimensional image of the heart using a localization system.

17. The system of claim 14, wherein the system is an electrophysiology monitoring system.

18. The system of claim 14, wherein the probe is a catheter configured to collect data representative of the electrical properties of the heart.

19. The system of claim 14, wherein the system is further configured to display a map of the electrical properties of the heart.

20. The system of claim 14, wherein the three-dimensional display is a spatial three-dimensional display.

21. A system for displaying a three-dimensional image of an organ or structure inside the body, the system comprising:
a processor configured to be communicatively coupled to a probe, the probe being configured to be located in or adjacent to the organ or structure inside the body and to collect data representative of the electrical properties of the organ or structure inside the body;
memory coupled to the processor and configured to store image data pertaining to the organ or structure inside the body; and
a three-dimensional display coupled to the processor and configured to display the three-dimensional image and a map of the electrical properties of the organ or structure inside the body.

22. The system of claim 21, wherein the display is further configured to simultaneously display a representation of the probe, wherein the representation of the probe is registered with the three dimensional image of the organ or structure inside the body.

23. A method of displaying a three-dimensional image of an organ or structure inside the body, the method comprising:
acquiring a three-dimensional image of the organ or structure inside the body;
registering a representation of a probe with the three-dimensional image, the probe being located in or adjacent to the organ or structure inside the body; and
simultaneously displaying a representation of the probe with the three-dimensional image using a three-dimensional display.

24. The method of claim 23, further comprising displaying a map of the electrical properties of the organ or structure inside the body.

25. The method of claim 23, wherein the organ or structure inside the body is a heart.

26. The method of claim 23, wherein the probe is a catheter.

27. The method of claim 23, further comprising displaying visual navigational information with the three-dimensional image and the representation of the probe.

28. The method of claim 27, wherein the visual navigational information includes changes in color indicate a proximity of the probe to a location or area of the three-dimensional image.

29. A system for displaying a three-dimensional image of an organ or structure inside the body, the system comprising:
memory configured to store a first set of image data pertaining to the organ or structure inside the body;
a processor coupled to the memory and configured to be communicatively coupled to an imaging device and a probe, the imaging device being configured to generate a second set of image data pertaining to the organ or structure inside the body, and the probe being configured to be located in or adjacent to the organ or structure inside the body, the processor further configured to generate the three-dimensional image using the first set of image data and the second set of image data; and
a three-dimensional display coupled to the processor and configured to simultaneously display the three-dimensional image and a representation of the probe.

30. The system of claim 29, wherein the system is configured to provide a warning related to an image-guided interventional procedure.

31. The system of claim 29, wherein the system is configured to provide a warning when the first set of image data differs from the second set of image data according to a predetermined criterion.

32. The system of claim 29, wherein the system is configured to determine a first estimate of the location of the probe and a second estimate of the location of the probe and to provide a warning when the first estimate differs from the second estimate according to a predetermined criterion.
